(19) United States
(12) Patent Application Publication
(10) Pub. No.: US 2024/0325659 A1
(43) Pub. Date: Oct. 3, 2024

(54) ADMINISTRATION OF NAKED NUCLEIC ACID MOLECULE

(71) Applicant: Daicel Corporation, Tokyo (JP)

(72) Inventors: Naoki SAKAGUCHI, Tokyo (JP); Kunihiko YAMASHITA, Tokyo (JP); Kazuhiro TERAI, Tokyo (JP)

(73) Assignee: Daicel Corporation, Tokyo (JP)

(21) Appl. No.: 18/575,953

(22) PCT Filed: Jul. 6, 2022
(86) PCT No.: PCT/IB2022/056252

§ 371 Date: Jan. 2, 2024

Related U.S. Application Data
(60) Provisional application No. 63/218,899, filed on Jul. 6, 2021.

(51) Int. Cl. A61M 5/48; A61K 9/00; A61K 48/00; A61M 5/20; A61M 5/30; C12N 15/87

(52) U.S. Cl. CPC A61M 5/482; A61K 9/0019; A61K 48/0016; A61M 5/2046; A61M 5/30; C12N 15/87

(57) ABSTRACT

Provided herein is a method of administering a naked nucleic acid molecule to a subject. The method may include injecting the naked nucleic acid molecule to the subject, wherein the injecting exhibits a bi-phasic injection profile comprising a first phase and a second phase, the second phase being after the first phase, and the bi-phasic injection profile has (i) at least two peaks within 15 msec from the injecting, (ii) the first peak of at least 2 MPa, or (iii) the highest peak of the second phase of the bi-phasic injection within 30 msec from the injecting.

### BACKGROUND

Non-viral gene delivery offers potential solutions to the limitations of viral-vector-based vaccines, as exemplified by the reports of optimized DNA-based gene-delivery systems developed over the past few decades. Naked DNA delivery, however, is significantly inhibited by the barriers of size, shape, and polyanionic charge of DNA, thus inhibiting the cell permeability of DNA and DNA susceptibility against serum nuclease. Direct injection of naked DNA plasmid in mice via the intramuscular, intradermal or intravenous routes enables the transfection of the gene of interest into muscle, skin and liver tissue, respectively, but the in vivo transfection efficiency of naked DNA is limited by its chemical instability, susceptibility to nuclease attack, rapid clearance and inefficient delivery to local lymph nodes. Cationic lipids have been widely used to form liposomal complexes with DNA for increased transfection, and new delivery systems such as transdermal patches can enhance the targeted delivery of DNA plasmids to skin-resident dendritic cells. Naked DNA delivery, however, is significantly inhibited by the barriers of size, shape, and polyanionic charge of DNA, thus inhibiting the cell permeability of DNA and DNA susceptibility against serum nuclease. Moreover, for mRNA-based vaccines, the chemical instability and low transfection efficacy of mRNA remain major barriers to therapeutic efficacy, and the in vivo delivery of naked mRNA remains challenging.

### SUMMARY OF THE DISCLOSURE

To solve the problem of instability of a naked nucleic acid molecule, the present disclosure provides, in one aspect, a method of administering a naked nucleic acid molecule to a subject, comprising injecting the naked nucleic acid molecule to the subject, wherein the injecting exhibits a bi-phasic injection profile having (i) at least two peaks within 15 msec from the injecting or (ii) the first peak of at least 2 MPa.

In another aspect, the present disclosure also provides a method of expressing a gene in a subject, comprising administering a naked nucleic acid molecule comprising the gene to the subject according to the method described herein.

In another aspect, the present disclosure further provides a method of treating, ameliorating or preventing a disease in a subject in need thereof, comprising expressing a gene in the subject in accordance with the method described herein, wherein the naked nucleic acid molecule triggers an antigen-specific immune response against the disease.

In another aspect, the present disclosure related to use of an injector for administering a naked nucleic acid molecule to a subject according to the method described herein. In another aspect, the present disclosure related to use of an injector for expressing a gene in a subject according to the method described herein. In another aspect, the present disclosure related to use of an injector for treating, ameliorating or preventing cancer in a subject in need thereof according to the method described herein.

### BRIEF DESCRIPTION OF THE DRAWINGS

FIG. 1A and FIG. 1B are diagrams showing an exemplary injection pressure transition.

FIG. 2 is a diagram showing respective transitions of combustion pressure related to powder combustion, pressure applied to a sealed dosing liquid, and injection pressure.

FIG. 3 depicts the effect of gene expression enhancement on GFP-encoded Naked mRNA by the injection described herein.

FIG. 4 depicts the gene expression enhancing effect on Luc-encoded Naked mRNA by the injection described herein.

FIG. 5A and FIG. 5B are diagrams showing a first alternative injection pressure transition.

FIG. 6A and FIG. 6B are diagrams showing a second alternative injection pressure transition.

### DETAILED DESCRIPTION

Hereinafter, exemplary embodiments of the present disclosure will be described in detail. However, the present disclosure is not limited to the embodiments disclosed below, but may be implemented in various forms. The following embodiments are described in order to enable those of ordinary skill in the art to embody and practice embodiments of the present disclosure.

### Definitions

Although the terms first, second, etc. may be used to describe various elements, these elements are not limited by these terms. These terms are only used to distinguish one element from another. For example, a first element could be termed a second element, and, similarly, a second element could be termed a first element, without departing from the scope of exemplary embodiments. The term “and/or” includes any and all combinations of one or more of the associated listed items.

The terminology used herein is for the purpose of describing particular embodiments only and is not intended to be limiting of exemplary embodiments. The singular forms “a,” “an” and “the” are intended to include the plural forms as well, unless the context clearly indicates otherwise.

The terms “comprises,” “comprising.” “includes” and/or “including.” used above, specify the presence of stated features, integers, steps, operations, elements, components and/or groups thereof, but do not preclude the presence or addition of one or more other features, integers, steps, operations, elements, components and/or groups thereof. All terminologies used herein including technical and scientific terminologies may hold the same meaning as are generally understood by those skilled in the art of technology of the present invention. Predefined, commonly used terminologies can hold the same or similar meaning to the contextual meaning of the relevant technology, and is not interpreted in an ideal or overly formal sense unless the context clearly indicates otherwise.

As used herein, the term “about” means modifying, for example, lengths of nucleotide sequences, degrees of errors, dimensions, the quantity of an ingredient in a composition, concentrations, volumes, process temperature, process time, yields, flow rates, pressures, and like values, and ranges thereof, refers to variation in the numerical quantity that may occur, for example, through typical measuring and handling procedures used for making compounds, compositions, concentrates or use formulations; through inadvertent error in these procedures; through differences in the manufacture, source, or purity of starting materials or ingredients used to carry out the methods; and like considerations. The term “about” also encompasses amounts that differ due to aging of, for example, a composition, formulation, or cell culture with a particular initial concentration or mixture, and amounts that differ due to mixing or processing a composition or formulation with a particular initial concentration or mixture. Whether modified by the term “about” the claims appended hereto include equivalents to these quantities. The term “about” further may refer to a range of values that are similar to the stated reference value. In certain embodiments, the term “about” refers to a range of values that fall within 50, 25, 10, 9, 8, 7, 6, 5, 4, 3, 2, 1 percent or less of the stated reference value.

### Overview

With reference to the appended drawings, exemplary embodiments of the present disclosure will be described in detail below. To aid in understanding the present disclosure, like numbers refer to like elements throughout the description of the figures, and the description of the same elements will be not reiterated.

In one aspect, the present disclosure provides a method of administering a naked nucleic acid molecule to a subject, comprising injecting the naked nucleic acid molecule to the subject, wherein the injecting exhibits a bi-phasic injection profile having (i) at least two peaks within 15 msec from the injecting or (ii) the first peak of at least 2 MPa.

“Naked” nucleic acid molecule refers to a nucleic acid molecule that is not associated with proteins, lipids, or any other molecule to help protect it. The naked nucleic acid molecule may be produced in the laboratory for use in, or as the result of, genetic engineering.

In certain embodiments, the naked nucleic acid molecule is a DNA. In certain embodiments, the naked nucleic acid molecule described herein excludes a DNA. DNA is the usual abbreviation for deoxy-ribonucleic acid. It is a nucleic acid molecule, i.e. a polymer consisting of nucleotides. These nucleotides are usually deoxy-adenosine-monophosphate, deoxy-thymidine-monophosphate, deoxy-guanosine-monophosphate and deoxy-cytidine-monophosphate monomers, which are—by themselves—composed of a sugar moiety (deoxyribose), a base moiety and a phosphate moiety, and polymerise to form a characteristic backbone structure. The backbone structure is, typically, formed by phosphodiester bonds between the sugar moiety of the nucleotide, i.e. deoxyribose, of a first and a phosphate moiety of a second, adjacent monomer. The specific order of the monomers, i.e. the order of the bases linked to the sugar/phosphate-backbone, is called the DNA sequence. DNA may be single stranded or double stranded. In the double stranded form, the nucleotides of the first strand typically hybridize with the nucleotides of the second strand, e.g. by A/T-base-pairing and G/C-base-pairing.

In certain embodiments, the naked nucleic acid molecule is an RNA. RNA is the usual abbreviation for ribonucleic-acid. It is a nucleic acid molecule, i.e. a polymer consisting of nucleotides. These nucleotides are usually adenosine-monophosphate, uridine-monophosphate, guanosine-monophosphate and cytidine-monophosphate monomers which are connected to each other along a so-called backbone. The backbone is formed by phosphodiester bonds between the sugar, i.e. ribose, of a first and a phosphate moiety of a second, adjacent monomer. The specific succession of the monomers is called the RNA sequence. Usually, RNA may be obtainable by transcription of a DNA sequence, e.g., inside a cell. In eukaryotic cells, transcription is typically performed inside the nucleus or the mitochondria. In vivo, transcription of DNA usually results in the so-called premature RNA, which has to be processed into so-called messenger RNA, usually abbreviated as mRNA. Processing of the premature RNA. e.g. in eukaryotic organisms, comprises a variety of different posttranscriptional-modifications such as splicing, 5′-capping, polyadenylation, export from the nucleus or the mitochondria and the like. The sum of these processes is also called maturation of RNA. The mature messenger RNA usually provides the nucleotide sequence that may be translated into an amino acid sequence of a particular peptide or protein. Typically, a mature mRNA comprises a 5′-cap, a 5′-UTR, an open reading frame, a 3′-UTR and a poly(A) sequence. Aside from messenger RNA, several non-coding types of RNA exist, which may be involved in the regulation of transcription and/or translation. The RNA may be selected from the group consisting of a small interfering RNA (siRNA), an asymmetrical interfering RNA (aiRNA), a microRNA (miRNA), a Dicer-substrate RNA (dsRNA), a small hairpin RNA (shRNA), a messenger RNA (mRNA), and mixtures thereof. In certain embodiments, the naked nucleic acid molecule is an mRNA. An mRNA may encode any peptide of interest, including any naturally or non-naturally occurring or otherwise modified peptide. A peptide encoded by an mRNA may be of any size and may have any secondary structure or activity. In additional embodiments, a peptide encoded by an mRNA may have a therapeutic effect when expressed in a cell. The RNA or mRNA described herein may include a first region of linked nucleosides encoding a peptide of interest (e.g., a coding region), a first flanking region located at the 5′-terminus of the first region (e.g., a 5′-UTR), a second flanking region located at the 3′-terminus of the first region (e.g., a 3′-UTR), at least one 5′-cap region, and a 3′-stabilizing region. In certain embodiments, the RNA or mRNA further includes a poly-A region or a Kozak sequence (e.g., in the 5′-UTR). In certain embodiments, the RNA or mRNA may include a 5′ cap structure, a chain terminating nucleotide, a stem loop, a polyA sequence, and/or a polyadenylation signal. Any one of the regions of the RNA or mRNA may include one or more alternative components (e.g., an alternative nucleoside). For example, the 3′-stabilizing region may contain an alternative nucleoside such as an L-nucleoside, an inverted thymidine, or a 2′-O-methyl nucleoside and/or the coding region, 5′-UTR, 3′-UTR, or cap region may include an alternative nucleoside such as a 5-substituted uridine (e.g., 5-methoxyuridine), a 1-substituted pseudouridine (e.g., 1-methyl-pseudouridine or 1-ethyl-pseudouridine), and/or a 5-substituted cytidine (e.g., 5-methyl-cytidine).

In some embodiments, the naked nucleic acid molecules described herein is a naked mRNA. In some embodiments, an amount of the naked mRNA administered is at least about 0.1, 0.2, 0.3, 0.4, 0.5, 0.6, 0.7, 0.8, 0.9, 1, 5, 10, 20, 30, 40, 50 or 60 μg. In additional embodiments, an amount of the naked mRNA administered is about 200, 190, 180, 170, 160, 150, 140, 130, 120, 110, 100, 90, 80, 70, 60, 50, 40, 30, 20, 10, 9, 8, 7, 6, 5, 4, 3, 2, 1 μg or less. In further embodiments, an amount of the naked mRNA administered is about from 0.2 μg to 150 μg, from 50 μg to 100 μg, from 10 μg to 150 μg, from 30 μg to 100 μg or from 20 μg to 110 μg.

Despite the recent progress of vaccine delivery systems, the in vivo delivery of the naked nucleic acid molecule remains challenging. For example, in the case of mRNA, the ribose sugar backbone of RNA, unlike the deoxyribose sugar backbone in DNA, is prone to hydrolysis, which reduces the stability of RNA molecules in circulation. Mammalian mRNAs are on average ˜2,000 nucleotides long, and a single event of hydrolysis along the mRNA backbone can impede its translation. Furthermore, ubiquitous ribonucleases within the body decrease the stability of RNA and reduce its therapeutic efficacy. By using the cartridge described herein, however, the personalized vaccines may remain viable without a modification to the DNA, RNA, or a vaccine composition thereof.

In certain embodiments, the naked nucleic acid molecule described herein is administered as a vaccine. The term “vaccine” means a biological preparation that induces or improves immunity against a particular disease. Typically, the vaccine comprises a conventional saline or buffered aqueous solution medium in which the composition of the present invention is suspended or dissolved. In this form, the composition of the present invention can be used conveniently to prevent, ameliorate, or otherwise treat a disease or disorder, such as infection. Upon introduction into a host, the vaccine is able to provoke an immune response including, but not limited to, the production of antibodies and/or cytokines and/or the activation of CD8+ T cells, antigen presenting cells, CD4+ T cells, dendritic cells and/or other cellular responses. In certain embodiments, the method comprises administering a vaccine comprising the naked nucleic acid molecule described herein. In some embodiments, the vaccine excludes a nanoparticle. In some embodiments, the vaccine excludes a cationic lipid. In some embodiments, the vaccine excludes a PEG lipid. In some embodiments, the vaccine excludes a phospholipid. In some embodiments, the vaccine excludes a lipid. In some embodiments, the vaccine includes an adjuvant. In some embodiments, the vaccine excludes an adjuvant. In certain embodiments, the adjuvant may be polyinosinic:polycytidylic acid (poly(I:C). In some embodiments, the vaccine excludes a DNA-encoded immunostimulatory gene. In some embodiments, the vaccine excludes a liposome. In some embodiments, the vaccine is non-viral. In some embodiments, the vaccine consists of the nucleic acid molecule and a buffer. In additional embodiments, the buffer may be saline. Vaccines can be made, for example, according to methods disclosed in WO2022112498A, WO2022049093A, or U.S. Pat. No. 10,913,964, the disclosure of which is hereby incorporated by reference.

In some embodiments, the method described herein excludes a nanoparticle. In some embodiments, the method excludes a cationic lipid. In some embodiments, the method excludes a lipid. In some embodiments, the method excludes an adjuvant. In some embodiments, the method excludes a DNA-encoded immunostimulatory gene. In some embodiments, the method excludes a liposome. In some embodiments, the method excludes a virus. In some embodiments, the naked nucleic acid molecule is injected only with a buffer.

As described herein, the instability of naked nucleic acid molecule is a challenge in expressing the nucleic acid molecule in a subject upon administration. The naked nucleic acid molecule may be taken up by endocytosis, and for example, the naked mRNA without the endosomal escape function of lipid nanoparticles (LNP) may not escape from endosomes, resulting in low expression of the gene. A method of injecting the naked nucleic acid molecule described herein may increase the expression of the DNA or RNA upon injection to the subject. The naked nucleic acid molecule may be delivered directly to cytosol of cells, resulting in high expression of the gene. In some embodiments, the injecting described herein exhibits a bi-phasic injection profile. The “bi-phasic injection profile” herein means that upon injection, at least two phases of injection pressure are measured over time. “A first phase of the bi-phasic injection profile” refers to the first phase measured, and “a second phase of the bi-phasic injection profile” refers to the second phase measured immediately after the first phase.

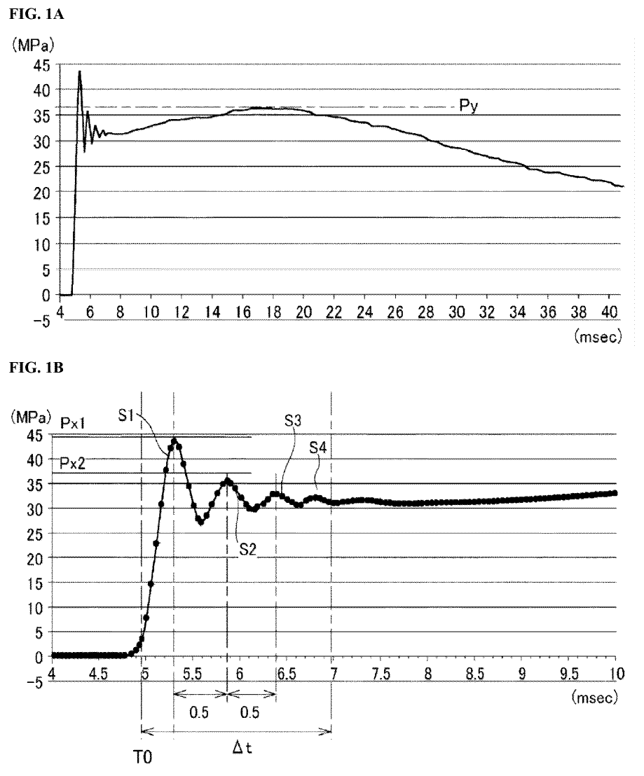

The bi-phasic injection profile may be accomplished by different pressure sources, for example, and an exemplary bi-phasic injection profile is as shown in FIGS. 1A and 1B.

FIG. 1A and FIG. 1B are injection profiles showing an exemplary transition of pressure (hereinafter, simply referred to as “injection pressure”) that can be applied to the naked nucleic acid molecule or the vaccine described herein. In FIG. 1A and FIG. 1B, an abscissa represents elapsed time in milliseconds (“msecs”) and an ordinate represents injection pressure in MPa. Moreover, injection pressure can be measured using conventional art. For example, in a similar manner to a measurement method described in Japanese Patent Application Laid-open No. 2005-21640, an injection force can be measured by a method involving applying force of an injection in a distributed manner to a diaphragm of a load cell arranged on a downstream side of a nozzle, sampling output from the load cell with a data sampling apparatus via a detection amplifier, and storing the sampled output as an injection force (N) per unit time. Injection pressure is calculated by dividing an injection force measured in this manner by an area of an injection port of an injector.

In certain embodiments, the transition of injection pressure and thus the injection profile may be modified by adopting different ignition charge materials in the igniter. For example, the ignition charge materials may include gunpowder (ZPP) containing zirconium and potassium perchlorate, gunpowder (THPP) containing titanium hydride and potassium perchlorate, gunpowder (TiPP) containing titanium and potassium perchlorate, gunpowder (APP) containing aluminum and potassium perchlorate, gunpowder (ABO) containing aluminum and bismuth oxide, gunpowder (AMO) containing aluminum and molybdenum oxide, gunpowder (ACO) containing aluminum and copper oxide, and gunpowder (AFO) containing aluminum and iron oxide, and gunpowder consisting of a combination of a plurality of these gunpowders. These gunpowders may exhibit characteristics in that, while high-temperature and high-pressure plasma is generated during combustion immediately after ignition, generated pressure drops abruptly once a combustion product reaches normal temperature and condenses since the combustion product does not have a gaseous component. Gunpowders other than the above may be used as the ignition charge material insofar as appropriate administration can be performed.

In certain embodiments, the transition of injection pressure and thus the injection profile may be modified by adopting different gas generating agents to be burned by a combustion product from the igniter to generate gas. The gas generating agent may be exposed to the combustion product from the igniter. The gas generating agents to be arranged inside the igniter is already well known as disclosed in WO 2001/031282 and Japanese Patent Application Laid-open No. 2003-25950. In addition, examples of the gas generating agent include a single-base smokeless powder consisting of 98% by mass of nitrocellulose, 0.8% by mass of diphenylamine, and 1.2% by mass of potassium sulfate. Furthermore, various gas generating agents used in an airbag gas generator or a seat-belt pretensioner gas generator can also be used. By adjusting dimensions or a size, a shape, and particularly a surface profile of a gas generating agent when arranging the gas generating agent, a combustion completion time of the gas generating agent can be varied and, accordingly, a pressure transition applied to the dosing liquid can be adjusted and a desired injection pressure transition of the dosing liquid can be achieved.

The bi-phasic injection profile described herein is not limited to the injection pressure profile generated by ignition. The bi-phasic injection profile described herein may be accomplished by other methods, for example, by controlling gas volume and/or speed applied to the naked nucleic acid molecule or the vaccine thereof.

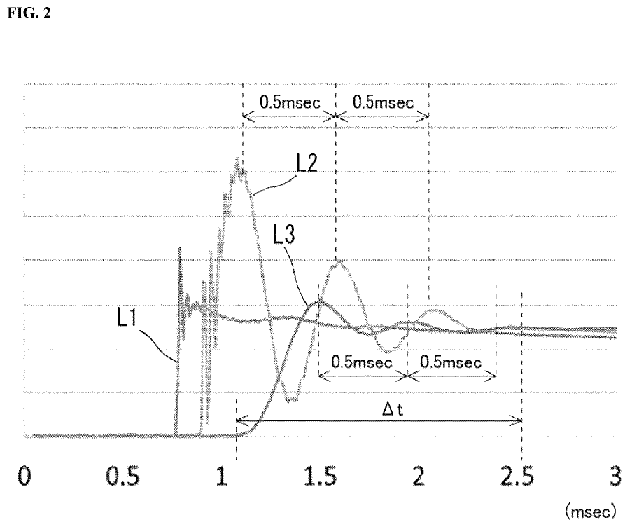

The injection profile shown in FIGS. 1A and 2B, for example, depicts the first phase based on an initial ignition of an ignition charge material, and the second phase having one peak based on a gas generating agent as described above. The first phase in this example comprises four vibration elements (i.e., S1 to S4), each having two local minimum values before and after a vibration peak. One vibration element ends at the later local minimum after the vibration peak.

In some embodiments, the bi-phasic injection profile has at least two peaks within about 15, 14, 13, 12, 11, 10, 9, 8, 7, 6, 5, 4.5, 4, 3.5, 3, 2.5, 2, 1.5, 1 or 0.5 msec from the injecting. The term “from the injecting” herein may mean starting from the time that a pressure is starting to be applied to the naked nucleic acid molecule or the vaccine thereof and/or the time that an increase of pressure on the naked nucleic acid molecule or the vaccine thereof is detected. In certain embodiments, the bi-phasic injection profile has the first peak within about 5, 4, 3, 2, 1, 0.9, 0.8, 0.7, 0.6, 0.5, 0.4, 0.3, 0.2 or 0.1 msec from the injecting.

In some embodiments, the bi-phasic injection profile described herein may comprise a first phase comprising a plurality of vibration elements, each having a vibration peak and two local minimum values before and after the vibration peak. In some embodiments, the total amplitudes of the vibration elements decrease over time. In some embodiments, the at least two peaks described above are vibration peaks. In some embodiments, the first peak of the bi-phasic injection profile described above is a vibration peak.

FIG. 1A represents an exemplary injection profile showing a transition of injection pressure during a period of approximately 40 msecs from start of combustion with a time point at which a start button on an injector is pressed, and FIG. 1B displays an enlargement of an injection pressure transition in an initial period (approximately 10 msecs from the origin) in the pressure transition shown in FIG. 1A. Moreover, rising of injection pressure occurs not at the origin but in a vicinity of 5 msecs because a certain amount of time is required for the ignition charge material to burn, the naked nucleic acid molecule or the vaccine thereof to be pressurized as a piston is propelled by the combustion energy of the ignition charge. In the exemplary injection pressure transition shown in FIGS. 1A and 1B, a plurality of pressure vibration elements S1 to S4 are present in a prescribed period of time Δt from the rise timing T0 to approximately 2 msecs thereafter, and pressure vibration generally converges once the prescribed period of time Δt elapses. Moreover, in the present embodiment, one cycle in which injection pressure rises and drops in pressure vibration is to be handled as one pressure vibration element.

In certain embodiments, the bi-phasic injection profile completes the first phase (e.g., the first phase is completed, for example, at the later local minimum of the last vibration element and/or at the start of the second phase) within about 15, 14, 13, 12, 11, 10, 9, 8, 7, 6, 5, 4, 3, 2, 1.9, 1.8, 1.7, 1.6, 1.5, 1.4, 1.3, 1.2, 1.1, 1.0, 0.9, 0.8, 0.7, 0.6, 0.5, 0.4, 0.3, 0.2 or 0.1 msec.

In FIGS. 1A and 1B, in the prescribed period of time Δt from the rise timing T0, a pressure vibration element S1 (hereinafter, referred to as a “first vibration element S1”) may be initially generated. The first vibration element S1 is an injection pressure transition of a period including a peak value Px1 (in this example, approximately 45 MPa) that starts from injection pressure (in this example, approximately 0 MPa) at the rise timing T0 and until a next local minimum value arrives. In addition, the total amplitude of the first vibration element S1 is approximately 45 MPa in this example. The first vibration element S1 is further followed by a second vibration element S2, a third vibration element S3, and a fourth vibration element S4. From the rise timing T0 to the last local minimum value of the vibration element(s) (e.g., the later local minimum value at the end of the last vibration element(s)) arrives is called the “first phase.” The second vibration element S2 is an injection pressure transition of a period including a peak value Px2 (in this example, approximately 37 MPa) from a timing of the end of the first vibration element S1 and until a next local minimum value arrives. From the end of the local minimum value at the end of the first vibration element to the next local minimum value arrives, including the peak value Px2, is called the “second vibration element.” In addition, the total amplitude of the second vibration element S2 from the lowest local minimum value to the peak of the second element is approximately 10 MPa in this example. With respect to the third vibration element S3 and the fourth vibration element S4, a period that defines each vibration element and a total amplitude of each vibration element are similar to those of the second vibration element S2 and, although a detailed description thereof will be omitted, the total amplitude of the third vibration element S3 and the total amplitude of the fourth vibration element S4 have decreased with the passage of time. In other words, in the prescribed period of time Δt, the pressure transition becomes a damped vibration with the passage of time, and after the lapse of the prescribed period of time Δt, the pressure transition enters a state where the vibration has more or less converged.

In some embodiments, the total amplitudes of the vibration elements of at least one phase of the bi-phasic injection profile decrease over time. In some embodiments, the total amplitudes of the vibration elements of the first phase of the bi-phasic injection profile decrease over time.

In some embodiments, the first peak of the bi-phasic injection profile described herein is at least about 0.5, 1, 2, 3, 4, 5, 6, 7, 8, 9, 10, 11, 12, 13, 14, 15 or 16 MPa. In some embodiments, the first peak of the bi-phasic injection profile described herein is below about 50, 49, 48, 47, 46, 45, 44, 43, 42, 41, 40, 39, 38, 37, 36, or 35 MPa.

The first peak of the bi-phasic injection profile may be the highest peak of the first phase of the bi-phasic injection. The first peak of the bi-phasic injection profile may be the highest vibration peak of the first phase of the bi-phasic injection, and the later vibration elements of the first phase may have peaks of lower heights, for example, as shown in FIGS. 1A and 1B. The height of the highest peak of the first phase and/or the height of the first peak of the bi-phasic profile may be predetermined or adjusted depending on the subject tissue to which the naked nucleic acid molecule or the vaccine thereof is administered. For direct administration to organs and vulnerable lesions, for example, the bi-phasic injection profile may have the highest peak of the first phase and/or the first peak of at least about 0.5, 1, 2, 3, 4 or 5 MPa. In additional embodiments, the bi-phasic injection profile has highest peak of the first phase and/or the first peak below about 20, 19, 18, 17, 16, 15, 14, 13, 12, 11, 10, 9, 8, 7, 6, or 5 MPa. In additional embodiments, the highest peak of the first phase and/or the first peak of the bi-phasic injection profile is from 0.5 MPa to 20 MPa, from 0.5 MPa to 15 MPa, or from 0.5 MPa to 5 MPa. For transdermal injection, for example, the bi-phasic injection profile has the highest peak and/or the first peak of at least about 15, 16, 17, 18, 19, 20, 21, 22, 23, 24, 25, 26, 27, 28, 29, 30, 31, 32, 33, 34, 35, 36, 37, 38, 39, 40, 41, 42, 43, 44 or 45 MPa. In additional embodiments, the bi-phasic injection profile has the highest peak of the first phase and/or the first peak below about 50, 49, 48, 47, 46, 45, 44, 43, 42, 41, 40, 39, 38, 37, 36, or 35 MPa. In additional embodiments, the highest peak of the first phase and/or the first peak of the bi-phasic injection profile is from 15 MPa to 50 MPa, from 30 MPa to 36 MPa, or from 20 MPa to 36 MPa.

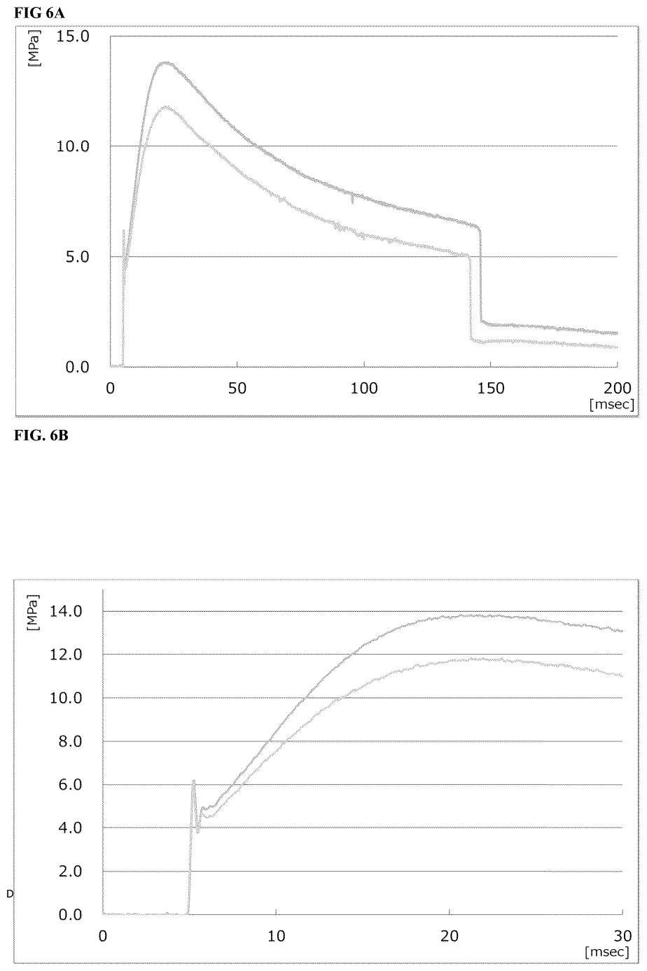

Alternatively, the highest peak in the bi-phasic injection profile may be in the second phase of the bi-phasic injection as illustrated in FIGS. 5A, 5B, 6A and 6B. The height of the highest peak of the first and second phases of the bi-phasic profile may be predetermined or adjusted depending on the subject tissue to which the vaccine is administered. For example, the bi-phasic injection profile may have the highest peak of the first phase of at least about 0.5, 1, 2, 3, 4, 5, 6, 7, 8, 9, 10, 11, 12, 12, 14, or 15 MPa. Moreover, the highest peak of the first phase may be less than 50, 45, 40, 39, 38, 37, 36, or 35 MPa. Furthermore, the bi-phasic injection profile may have the highest peak of the second phase of at least about 10, 12, 14, 16, 20, 21, 22, 23, 24, 25, 26, or 27 MPa. Moreover, the highest peak of the first phase may be less than 80, 75, 70, 68, 66, 65, 64, 63, 62, 61, or 60 MPa.

In certain embodiments, a period calculated from a peak value of the first vibration element S1 to a peak value of the second vibration element S2 is within about 1, 0.9, 0.8, 0.7, 0.6, 0.5, 0.4, or 0.3 msec. In certain embodiments, a period calculated from the peak value of the second vibration element S2 to a peak value of the third vibration element S3 is within about 1.1, 1, 0.9, 0.8, 0.7, 0.6, 0.5, 0.4, or 0.3 msecs. While a period immediately before arrival of a converged state may be slightly shorter, the transition of injection pressure may take place at a generally constant period in the prescribed period of time Δt. In certain embodiments, the injection pressure transition in the prescribed period of time Δt may be a pressure vibration at a frequency of around 2200, 2100, 2000, 1900, 1800 or 1700 Hz or less. In certain embodiments, the pressure vibration may be at a frequency of around 1500, 1600, 1700, 1800, 1900, or 2000 Hz or more. When the second phase of the bi-phasic injection profile is higher than the first phase, it may not be necessary to have vibration in injection pressure during the first phase of the bi-phasic profile. For example, when the pressure at the highest peak of the second phase is 2, 3, 4, 5, 6, or 7 times larger than pressure at the highest peak of the first phase, the vibration of the first phase is not necessary for injection of the vaccine. Likewise, it may not be necessary to have vibration in injection pressure during the first phase of the bi-phasic injection profile when the first phase has a higher peak than the second phase.

In certain embodiments, the pressure fluctuation in the prescribed period of time Δt may be attributable to combustion of the ignition charge material of the igniter described herein. In addition, in a vicinity of a timing at which the prescribed period of time Δt lapses, combustion of a gas generating agent in the injector may be started by a combustion product of the ignition charge material and combustion energy thereof starts to further act on the naked nucleic acid molecule or the vaccine thereof. As a result, in the example shown in FIG. 1A, after the lapse of the prescribed period of time Δt, injection pressure increases one again and a peak value Py, which is called the “highest peak of the second phase,” arrives at a timing of approximately 18 msec. Moreover, subsequently, the injection pressure gradually drops with the passage of time. Since a combustion rate of the gas generating agent may be lower than a combustion rate of the ignition charge material, a rate of increase of injection pressure due to combustion of the gas generating agent also may become relatively lower. In certain embodiments, the combustion of the gas generating agent may start before about 8, 7.5, 7, 6.5, 6, 5.5, 5, 4.5, 3, 3.5, 3, 2.5, 2, 1.5 or 1 msec from the injecting. In certain embodiments, the peak Py for the combustion of the gas generating agent or the highest peak of the second phase may appear before about 30, 29, 28, 27, 26, 25, 24, 23, 22, 21, 20, 19, 18, 17, 16, 15, 14, 13, 12, 11, or 10 msec from the injecting. In certain embodiments, the peak Py for the combustion of the gas generating agent or the highest peak of the second phase may appear after about 7, 8, 9, 10, 11, 12, 13, 14, 15, 16, 17, 18, 19 or 20 msec from the injecting. In certain embodiments, the bi-phasic injection profile has at least one peak of the second phase before about 30, 29, 28, 27, 26, 25, 24, 23, 22, 21, 20, 19, 18, 17, 16, 15, 14, 13, 12, 11, 10, 9, 8, 7, 6, 5 or 4 msec from the injecting. In certain embodiments, the bi-phasic injection profile has at least one peak of the second phase after about 7, 8, 9, 10, 11, 12, 13, 14, 15, 16, 17, 18, 19 or 20 msec from the injecting.

In some embodiments, the bi-phasic injection profile may comprise a second phase having only one peak.

The height of the highest peak of the second phase of the bi-phasic profile may be predetermined or adjusted depending on the subject tissue to which the naked nucleic acid molecule or the vaccine thereof is administered. In certain embodiments, for direct administration to organs and vulnerable lesions, the highest peak of the second phase of the bi-phasic injection profile is at least about 0.1, 0.2, 0.3, 0.4, 0.5, 0.6, 0.7, 0.8, 0.9, 1, 2, 3, 4, 5, 6, 7, 8, 9, or 10 MPa. In additional embodiments, the highest peak of the second phase of the bi-phasic injection profile is below about 15, 14, 13, 12, 11, 10, 9, 8, 7, 6, 5, 4, 3, 2 or 1 MPa. In additional embodiments, the bi-phasic injection profile has the second peak about from 0.1 MPa to 15 MPa, from 1 MPa to 10 MPa, or from 3 MPa to 6 MPa. In certain embodiments, for transdermal injection, the highest peak of the second phase of the bi-phasic injection profile is at least about 20, 21, 22, 23, 24, 25, 26, 27, 28, 29, 30, 31, 32, 33, 34 or 35 MPa. In additional embodiments, the highest peak of the second phase of the bi-phasic injection profile is below about 45, 44, 43, 42, 41, 40, 39, 38, 37, 36, 35, 34, 33, 32, 31, 30 or 29 MPa. In additional embodiments, the highest peak of the second phase of the bi-phasic injection profile is from 30 MPa to 40 MPa, from 30 MPa to 36 MPa, or from 20 MPa to 36 MPa. In a further embodiment, the highest peak of the first phase may range from 10.0 MPa to 38.0 MPa, and the highest peak of the second phase may range from 25.0 MPa to 64.0 MPa.

In some embodiments, the highest peak of a second phase of the bi-phasic profile is lower than the highest peak of a first phase of the bi-phasic profile. In certain embodiments, the highest peak of a second phase of the bi-phasic profile is lower than the first peak of the first phase of the bi-phasic profile. In some embodiments, the highest peak of a first phase of the bi-phasic profile is lower than the highest peak of a second phase of the bi-phasic profile. In certain embodiments, the highest peak of a second phase of the bi-phasic profile is higher than the first peak of the first phase of the bi-phasic profile.

In certain embodiments, the injection is completed within about 400, 450, 300, 250, 200, 150, or 100 msec from the injecting.

In some embodiments, the injection is transdermal injection. In some embodiments, the injecting excludes transdermal injection.

In some embodiments, the injection is intramuscular. In some embodiments, the injection is subcutaneous. In some embodiments, the injection is intradermal. In some embodiments, the injection is intralesional. In certain embodiments, the naked nucleic acid molecule or the vaccine thereof may be injected to a particular organ of interest, for example during a surgery. In some embodiments, the injection is intratumoral. In some embodiments, the injection is the injection is intranodal. In some embodiments, excludes intranodal injection. In some embodiments, the injection is intralymphatic.

In some embodiments, the naked nucleic acid molecule is injected with a needleless injector. In some embodiments, the naked nucleic acid molecule is injected with an injector comprising an igniter. In some embodiments, the naked nucleic acid molecule is injected with an injector excluding a spring. In certain embodiments, the injector may be a needleless injector, the needleless injector comprising: the cartridge described herein, an igniter including an igniter powder which exhibits such a pressure characteristic that a plasma is generated during combustion immediately after ignition and then a generated pressure is lowered when a temperature becomes ordinary temperature and a combustion product is condensed on account of no gas component which is contained in the combustion product or any gas component which is contained in the combustion product and an amount of which is decreased as compared with that provided before the condensation; and a nozzle unit having a discharge port through which the naked nucleic acid molecule or the vaccine thereof pressurized by the combustion of the igniter powder in the igniter flows so that the naked nucleic acid molecule or the vaccine thereof is discharged to the injection target area. In additional embodiments, a temperature of the combustion product, which is provided during the pressurization, changes to a neighborhood of the ordinary temperature within 20 msec after a pressure, which is applied to the naked nucleic acid molecule or the vaccine thereof on account of the combustion of the igniter powder, reaches an initial peak discharge force during a pressurization process for discharging the naked nucleic acid molecule or the vaccine thereof. In additional embodiments, the temperature of the combustion product, which is provided during the pressurization, changes to the neighborhood of the ordinary temperature within 10 msec after the pressure, which is applied to the DNA solution on account of the combustion of the igniter powder, reaches the initial peak discharge force. In certain embodiments, the injector may be an injector that injects the naked nucleic acid molecule or the vaccine thereof into an injection target from an injector main body without performing injection through a given structure in a state where the given structure is inserted into the injection target. In additional embodiments, the injector comprises the cartridge, and a nozzle unit including an injection port through which the solution containing biomolecules flows and is injected into the injection target, the solution being pressurized by combustion of an ignition charge in an igniter. In additional embodiments, a maximum injection speed of the solution containing biomolecules between an injection start time of the solution containing biomolecules and a time of 0.20 ms is from 75 m/s to 150 m/s and an injection speed of the solution containing biomolecules of from 75 m/s to 150 m/s lasts for 0.11 ms or longer.

In certain embodiments, an exemplary injector and methods of using the injector may be those described in US Patent Application Publication Nos. 2018/0168789, 2018/0369484 and/or 2021/0023302, all of which are incorporated herein by reference.

In certain embodiments, the subject described herein is a human. In certain embodiments, the subject described herein is non-human. In certain embodiments, the subject described herein is a rodent. In certain embodiments, the subject described herein is mammal, bird, reptile, fish, amphibian, or invertebrate.

In one aspect, the present disclosure also provides a method of expressing a gene in a subject, comprising administering a naked nucleic acid molecule comprising the gene to the subject according to the method described herein. In some embodiments, the method of expressing a gene further comprises detecting expression of the gene in the subject in 6, 5, 4, or 3 hours or less from the injecting.

In one aspect, the present disclosure further provides a method of treating, ameliorating or preventing a disease in a subject in need thereof, comprising expressing a gene in the subject in accordance with the method described herein, wherein the naked nucleic acid molecule triggers an antigen-specific immune response against the disease.

In certain embodiments, the subject has a disease related to a mutation. The disease herein may include a disorder caused by a genetic mutation. In additional embodiments, and the naked nucleic acid molecule described herein comprises the mutation. In certain embodiments, the naked nucleic acid molecule described herein expresses an antigen. In the context of the present invention, “antigen” refers typically to a substance which may be recognized by the immune system, preferably by the adaptive immune system, and is capable of triggering an antigen-specific immune response, e.g. by formation of antibodies and/or antigen-specific T cells as part of an adaptive immune response. Typically, an antigen may be or may comprise a peptide or protein, which may be presented by the MHC to T-cells. In the sense of the present invention, an antigen may be the product of translation of a provided nucleic acid molecule as defined herein. In this context, also fragments, variants and derivatives of peptides and proteins comprising at least one epitope are understood as antigens. In certain embodiments, the naked nucleic acid molecule described herein expresses an antigen selected from the group consisting of a pathogenic antigen, a tumor antigen, an allergenic antigen and an autoimmune antigen. The antigen may be derived from a pathogen associated with an infectious disease. The antigen may be selected from the group consisting of a bacterial, a viral, a fungal and a protozoan pathogen.

In some embodiments, the disease or disorder is a cancer. In some embodiments, the disease or disorder is a tumor.

In some embodiments, the subject has a tumor. In some embodiments, the naked nucleic acid molecule triggers an antigen-specific immune response in a tumor. In additional embodiments, the naked nucleic acid molecule comprises a tumor-specific mutation. In certain embodiments, the antigen naked nucleic acid molecule may be a neoantigen nucleic acid molecule. In some embodiments, the naked nucleic acid molecule is neoantigen mRNA specific to a tumor. Genetic instability of tumor cells may lead to the occurrence of mutations, and expression of non-synonymous mutations may produce tumor-specific antigens called neoantigens. Neoantigens are highly immunogenic as they are not expressed in normal tissues. They can activate CD4+ and CD8+ T cells to generate immune response and have the potential to become new targets of tumor immunotherapy. The development of bioinformatics technology has accelerated the identification of neoantigens, and various neoantigens have been identified. Castle, J. C. et al. Exploiting the Mutanome for Tumor Vaccination, Cancer Res. 72, 1081-1091 (2012); Yadav, M. et al. Predicting immunogenic tumour mutations by combining mass spectrometry and exome sequencing, Nature 515, 572-576 (2014); Gubin, M. M. et al. Checkpoint blockade cancer immunotherapy targets tumour-specific mutant antigens, Nature 515, 577-581 (2014); Kreiter, S. et al. Mutant MHC class II epitopes drive therapeutic immune responses to cancer, Nature 520, 692-696 (2015); Ott, P. A. et al. An immunogenic personal neoantigen vaccine for patients with melanoma, Nature 547, 217-221 (2017); Sahin, U. et al. Personalized RNA mutanome vaccines mobilize poly-specific therapeutic immunity against cancer, Nature 547, 222-226 (2017); Keskin, D. B. et al. Neoantigen vaccine generates intratumoral T cell responses in phase Ib glioblastoma trial, Nature 565, 234-239 (2019); Hilf, N. et al. Actively personalized vaccination trial for newly diagnosed glioblastoma, Nature 565, 240-245 (2019). In some embodiments, the antigen naked nucleic acid molecule may be a neoantigen mRNA.

In certain embodiments, the antigen naked nucleic acid molecule may be a neoantigen naked nucleic acid molecule, and the cartridge may further contain an additional vaccine including patient-derived dendritic cell (DC) or a synthetic long peptide (SLP). In some embodiments, the antigen naked nucleic acid molecule may be a neoantigen mRNA. Cellular therapies based on patient-derived DCs (e.g., obtained from the ex vivo differentiation of peripheral blood monocytes) loaded with tumor-associated antigens (TAAs) may be infused back into the patient to enhance T-cell activation and tumor-cell killing. In some embodiments, the cartridge further comprises a blocking antibody specific for an immune checkpoint protein. In some embodiments, the immune checkpoint protein comprises cytotoxic T lymphocyte-associated antigen-4 (CTLA-4) and/or programmed cell death receptor-1 (PD-1). These antibodies, designed to liberate T cells from the immunosuppression mediated by the CTLA-4 and PD-1 pathways, may promote potent and durable T-cell responses that can eliminate tumors and lead to cancer remission.

In some embodiments, the disease or disorder is a virus infection. In some embodiments, the naked nucleic acid molecule described herein is mRNA to encode a viral protein. In some embodiments, the virus infection comprises coronavirus infection. In some embodiments, the naked nucleic acid molecule is mRNA to encode a coronavirus spike protein.

In certain embodiments, the subject is in need of a vaccine comprising a naked nucleic acid molecule against an infectious disease. In some embodiments, the vaccine triggers an antigen-specific immune response against coronavirus, including, but not limited to, Sars-COV2. In certain embodiments, the vaccine is a cytomegalovirus (CMV) vaccine, for example, including, but not limited to, the mRNA described in John, S. et al. Multi-antigenic human cytomegalovirus mRNA vaccines that elicit potent humoral and cell-mediated immunity, Vaccine 36(12), 1689-1699 (2018).

In one aspect, the present disclosure related to use of an injector for administering a naked nucleic acid molecule to a subject according to the method described herein. In another aspect, the present disclosure related to use of an injector for expressing a gene in a subject according to the method described herein. In another aspect, the present disclosure related to use of an injector for treating, ameliorating or preventing cancer in a subject in need thereof according to the method described herein.

### Examples

### Materials

Dulbecco-phosphate buffered aqueous solution (manufactured by Nacalai Tesque, D-PBS)
TE Buffer pH 8.0 (manufactured by Nacalai Tesque)
PBS-Tablet (manufactured by Takara Bio Inc.)
Water (manufactured by Nacalai Tesque)
Naked mRNA_GFP (CleanCap® EGFP mRNA manufactured by TriLink)
Naked mRNA_Luc (TriLink, CleanCap® FLuc mRNA)
Naked mRNA_U modification_Luc (TriLink, CleanCap® FLuc mRNA (5moU))
Passive Lysis Buffer 5× (manufactured by Promega)
Luciferase Assay (Promega, Luciferase Assay System)
C57BL/6 mice and BALB/c mice were purchased from Claire Japan.

### Devices Used

Cooling centrifuge (MDX-300 manufactured by Tomy)
Autoclave (manufactured by Tomy, LSX-700)
8 mm biopsy pouch (KAI industry, 8 mm Biopsy punch)
Luminometer (KIKKOMAN, C-100N)

First, whether or not the gene expression of the administered mRNA was enhanced by administering the mRNA to a living body using the device was evaluated in the GFP-encoding mRNA (Naked mRNA_GFP). For administration, 10-week-old male BALB/c mice were used, and euthanasia and data collection were performed six hours after administration. The device used for administration was a container having a nozzle diameter of 0.1 mm, 30 mg of ZPP ignition material, and 30 mg of GG gas generation material. The dose per administration was 20 μL, and the mRNA was 0.01 to 0.5 mg/mL (0.2 to 10 μg/shot). For gene expression, the skin at the administration site was attached to a Matsunami glass bottom dish and observed with a fluorescence microscope BZ-X710 manufactured by KEYENCE.

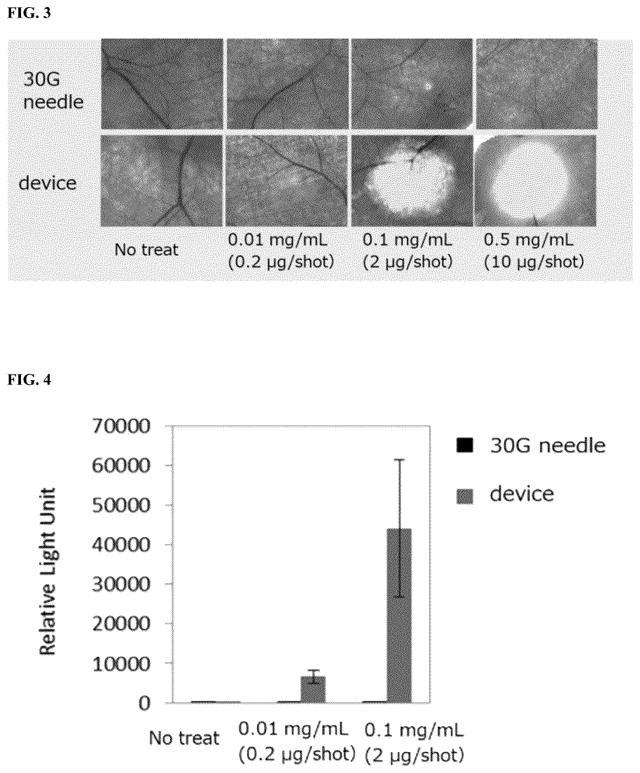

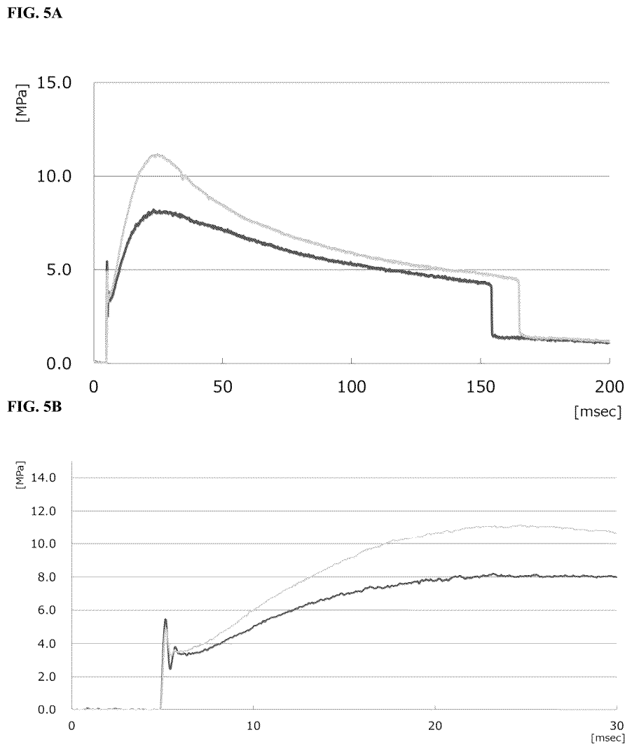

As a result, when administered using a 30 G needle, only a small amount of gene expression was obtained at any amount of mRNA. On the other hand, when administered using the device, gene expression was confirmed in the administered skin at any mRNA amount of 0.01 to 0.5 mg/mL (FIG. 3). The fluorescence intensity corresponding to the gene expression level increased depending on the amount of mRNA used, and it was confirmed that the administered mRNA was gene-expressed. Since the gene expression of naked mRNA by the device was confirmed even in a small amount of only 0.01 mg/mL, efficient mRNA cytosol delivery using the device and subsequent gene expression were obtained.

When the fluorescence intensity of GFP was compared between the 30 G needle and the device, the one using the device clearly had strong fluorescence at any mRNA amount of 0.01 to 0.5 mg/mL (FIG. 3). From these results, it was confirmed that the device is more likely to induce the gene expression of naked mRNA than the needle, and the gene-expression enhancing effect using the device was confirmed.

(2) Effect of Device Gene Expression Enhancement on Luc-Encoding mRNA

The gene-expression enhancing effect of the device was evaluated for mRNA encoding Luc (Naked mRNA_Luc), which was a different protein. For administration, 10-week-old male BALB/c mice were used, and euthanasia and data collection were performed six hours after administration. The device used for administration was a container having a nozzle diameter of 0.1 mm, 30 mg of ZPP ignition material, and 30 mg of GG gas generation material. The dose per administration was 20 μL, and the mRNA was 0.01 to 0.1 mg/mL (0.2 to 2 μg/shot). For gene expression, an 8 mm biopsy pouch was used to sample the skin at the administration site, and a 5-fold diluted Passive Lysis Buffer 5× was used to prepare lysate. Then, using the Luciferase Assay System manufactured by Promega and the Luminometer C-100N manufactured by Kikkoman, the amount of luciferase emitted for 10 seconds was measured to evaluate gene expression.

As a result, as in the case of GFP, when administered using a 30 G needle, only a small amount of gene expression was obtained at any mRNA amount. On the other hand, high gene expression was confirmed in those administered using the device (FIG. 4). In order to evaluate the gene-expression enhancing effect by the device, when the gene-expression level was compared between the 30 G needle and the device, the device was about 2,300 times higher for 0.01 mg/mL mRNA and about 300 times higher for 0.1 mg/mL mRNA (FIG. 4). The gene-expression enhancing effect by using the device was also confirmed in the Luc-encoding mRNA.

Thus, since the gene-expression enhancing effect of the device was confirmed in multiple reporter proteins such as GFP and Luc, it was clarified that the gene expression by the device was independent of the gene sequence encoded by the mRNA. From this, it is considered that the gene-expression enhancing effect of the device can be obtained with mRNA encoding any genes.

### (3) Alternate Injection Pressure Profile of Injector—Example 1

An injector having a nozzle diameter of 0.5 mm was filled with 150 μL of water, and the injection pressure in the injector from when pressurization of water was performed by combustion of an ignition charge until after injection was evaluated. Regarding the explosive, 55 mg of an explosive containing zirconium and potassium perchlorate (ZPP) was used, and regarding the gas generating agent, 40 mg of a single base smokeless explosive (hereinafter referred to as “GG” in some cases) was used.

For measurement of the injection pressure, like the measurement method in Japanese Patent Application Publication No. 2005-21640, a method in which an injection force was distributed and applied to a diaphragm of a load cell arranged downstream from a nozzle, an output from the load cell was collected in a data collection display device via a detection amplifier, and displayed and stored as an injection force (N) for each time was used for measurement, and the injection pressure was calculated by dividing the injection force (N) by an area of a nozzle port. The measurements were obtained using CLS-2NA from Tokyo Measuring Instruments Laboratory Co. Ltd. In total, 30 measurements were made.

Of the 30 measurements, the two measurements in which the highest and lowest peaks of the second phase of the bi-phasic profile were detected are illustrated in FIGS. 5A and 5B. The peak of the second phase was higher in all 30 measurements, with the average peak pressures of the first and second phases being 4.574 MPa and 9.598 MPa, respectively. On average, the peaks for the first and second phases were detected at 5.230 msec and 24.150 msec after ignition.

### (3) Alternate Injection Pressure Profile of Injector—Example 2

The same conditions used in Example 1 above were repeated except that the amounts of ZPP and GG were both increased from 55 mg to 65 mg. A total of 30 measurements were made and the two measurements in which the highest and lowest peaks of the second phase of the bi-phasic profile were detected are illustrated in in FIGS. 6A and 6B. The peak of the second phase was higher in all 30 measurements, with the average peak pressures of the first and second phases being 6.102 MPa and 12.562 MPa, respectively. On average, the peaks of the first and second phases were detected at 5.243 msec and 21.957 msec after ignition.

The following are some exemplary embodiments of the present disclosure.
Embodiment 1. A method of administering a naked nucleic acid molecule to a subject, comprising injecting the naked nucleic acid molecule to the subject, wherein the injecting exhibits a bi-phasic injection profile comprising a first phase and a second phase, the second phase being after the first phase, and the bi-phasic injection profile has (i) at least two peaks within 15 msec from the injecting, (ii) the first peak of at least 2 MPa, or (iii) the highest peak of the second phase of the bi-phasic injection within 30 msec from the injecting.
Embodiment 2. The method according to embodiment 1, wherein the bi-phasic injection profile has at least two peaks within 15 msec from the injecting.
Embodiment 3. The method according to embodiment 1 or 2, wherein the bi-phasic injection profile has at least two peaks within 1.5 msec from the injecting.
Embodiment 4. The method according to any one of the preceding embodiments, wherein the bi-phasic injection profile has the first peak within 5 msec.
Embodiment 5. The method according to any one of the preceding embodiments, wherein the first phase comprises a plurality of vibration elements, each having a vibration peak.
Embodiment 6. The method according to embodiment 5, wherein said at least two peaks are the vibration peaks of the vibration elements.
Embodiment 7. The method according to embodiment 5 or 6, wherein total amplitudes of said vibration elements decrease over time.
Embodiment 8. The method according to any one of the preceding embodiments, wherein the first peak is at least 2 MPa.
Embodiment 9. The method according to any one of the preceding embodiments, wherein the first peak is at least 15 MPa.
Embodiment 10. The method according to any one of the preceding embodiments, wherein the highest peak of the second phase of the bi-phasic injection profile is within 30 msec from the injecting.
Embodiment 11. The method according to any one of the preceding embodiments, wherein the highest peak of the second phase of the bi-phasic injection profile is within 15 msec from the injecting.
Embodiment 12. The method according to any one of the preceding embodiments, wherein the bi-phasic injection profile comprises the second phase having only one peak.
Embodiment 13. The method according to any one of the preceding embodiments, wherein the highest peak of the second phase of the bi-phasic profile is at least 0.1 MPa.
Embodiment 14. The method according to any one of the preceding embodiments, wherein the highest peak of the second phase of the bi-phasic profile is at least 10 MPa.
Embodiment 15. The method according to any one of the preceding embodiments, wherein the highest peak of the second phase of the bi-phasic profile is lower than the highest peak of a first phase of the bi-phasic profile.
Embodiment 16. The method according to any one of the preceding embodiments, wherein the highest peak of the second phase of the bi-phasic profile is higher than the highest peak of a first phase of the bi-phasic profile.
Embodiment 17. The method according to any one of the preceding embodiments, wherein the injection is transdermal injection.
Embodiment 18. The method according to any one of embodiments 1-16, wherein the injecting excludes transdermal injection.
Embodiment 19. The method according to any one of embodiments 1-18, wherein the injection is intramuscular, subcutaneous, or intradermal.
Embodiment 20. The method according to any one of embodiments 1-18, wherein the injection is intralesional.
Embodiment 21. The method according to any one of embodiments 1-18, wherein the injection is intratumoral.
Embodiment 22. The method according to any one of embodiments 1-18, wherein the injection is intranodal or intralymphatic.
Embodiment 23. The method according to any one of embodiments 1-21, wherein the injection excludes intranodal injection.
Embodiment 24. The method according to any one of the preceding embodiments, wherein the method excludes a nanoparticle.
Embodiment 25. The method according to any one of the preceding embodiments, wherein the method excludes a cationic lipid.
Embodiment 26. The method according to any one of the preceding embodiments, wherein the method excludes a lipid.
Embodiment 27. The method according to any one of the preceding embodiments, wherein the method excludes an adjuvant.
Embodiment 28. The method according to any one of the preceding embodiments, wherein the method excludes a DNA-encoded immunostimulatory gene.
Embodiment 29. The method according to any one of the preceding embodiments, wherein the method excludes a liposome.
Embodiment 30. The method according to any one of the preceding embodiments, wherein the method excludes a virus.
Embodiment 31. The method according to any one of the preceding embodiments, wherein the naked nucleic acid molecule is injected only with a buffer.
Embodiment 32. The method according to any one of the preceding embodiments, wherein the naked nucleic acid molecule triggers an antigen-specific immune response in a tumor.
Embodiment 33. The method according to any one of the preceding embodiments, wherein the naked nucleic acid molecule is mRNA.
Embodiment 34. The method according to embodiment 33, wherein an amount of the mRNA injected to the subject is at least 0.2 μg.
Embodiment 35. The method according to any one of the preceding embodiments, wherein the naked nucleic acid molecule is neoantigen mRNA specific to a tumor.
Embodiment 36. The method according to any one of embodiments 1-34, wherein the naked nucleic acid molecule is mRNA to encode a viral protein.
Embodiment 37. The method according to embodiment 36, wherein the naked nucleic acid molecule is mRNA to encode a coronavirus spike protein.
Embodiment 38. The method according to any one of embodiments 1-32, wherein the naked nucleic acid molecule is DNA.
Embodiment 39. The method according to any one of the preceding embodiments, wherein the naked nucleic acid molecule is injected with a needleless injector.
Embodiment 40. The method according to any one of the preceding embodiments, wherein the naked nucleic acid molecule is injected with an injector comprising an igniter.
Embodiment 41. The method according to any one of the preceding embodiments, wherein the naked nucleic acid molecule is injected with an injector excluding a spring.
Embodiment 42. A method of expressing a gene in a subject, comprising administering a naked nucleic acid molecule comprising the gene to the subject according to the method of any one of the preceding embodiments.
Embodiment 43. The method according to embodiment 42, further comprising detecting expression of the gene in the subject in 6 hours or less from the injecting.
Embodiment 44. A method of treating, ameliorating or preventing a disease or disorder in a subject in need thereof, comprising expressing a gene in the subject in accordance with the method of embodiment 42 or 43, wherein the naked nucleic acid molecule triggers an antigen-specific immune response against the disease or disorder.
Embodiment 45. The method according to embodiment 44, wherein the disease or disorder is a cancer.
Embodiment 46. The method according to embodiment 44, wherein the disease or disorder is a tumor.
Embodiment 47. The method according to embodiment 44, wherein the disease or disorder is a virus infection.
Embodiment 48. The method according to embodiment 47, wherein the virus infection comprises coronavirus infection.
Embodiment 49. Use of an injector for administering a naked nucleic acid molecule to a subject according to the method of any one of embodiments 1-41.
Embodiment 50. Use of an injector for expressing a gene in a subject according to the method of embodiment 42 or 43.
Embodiment 51. Use of an injector for treating, ameliorating or preventing cancer in a subject in need thereof according to the method of any one of embodiments 44-48.

1. A method of administering a naked nucleic acid molecule to a subject, comprising
injecting the naked nucleic acid molecule to the subject,
wherein the injecting exhibits a bi-phasic injection profile comprising a first phase and a second phase, the second phase being after the first phase, and
the bi-phasic injection profile has (i) at least two peaks within 15 msec from the injecting, (ii) the first peak of at least 2 MPa, or (iii) the highest peak of the second phase of the bi-phasic injection within 30 msec from the injecting.

2. The method according to claim 1, wherein the bi-phasic injection profile has at least two peaks within 15 msec from the injecting.

3. The method according to claim 1, wherein the bi-phasic injection profile has at least two peaks within 1.5 msec from the injecting.

4. The method according to claim 1, wherein the bi-phasic injection profile has the first peak within 5 msec.

5. The method according to claim 1, wherein the first phase comprises a plurality of vibration elements, each having a vibration peak.

6. The method according to claim 5, wherein total amplitudes of said vibration elements decrease over time.

7. The method according to claim 1, wherein the first peak is at least 2 MPa.

8. The method according to claim 1, wherein the highest peak of the second phase of the bi-phasic profile is at least 0.1 MPa.

9. The method according to claim 1, wherein the highest peak of the second phase of the bi-phasic profile is lower than the highest peak of a first phase of the bi-phasic profile.

10. The method according to claim 1, wherein the highest peak of the second phase of the bi-phasic profile is higher than the highest peak of a first phase of the bi-phasic profile.

11. The method according to claim 1, wherein the naked nucleic acid molecule is injected only with a buffer.

12. The method according to claim 1, wherein the naked nucleic acid molecule is mRNA.

13. The method according to claim 12, wherein the mRNA is injected in an amount of at least 0.2 μg.

14. The method according to claim 1, wherein the naked nucleic acid molecule is injected with a needleless injector.
